(19) United States
(12) Plant Patent Application Publication
(10) Pub. No.: US 2012/0240298 P1
(43) Pub. Date: Sep. 20, 2012

(54) Styrax plant named jlweeping

(50) ;

(76) Inventor: John Lewis, Salem, OR (US)

(21) Appl. No.: 13/065,105

(22) Filed: Mar. 14, 2011

(51) Int. Cl. A01H 5/00

(52) U.S. Cl. PLT/216

(57) ABSTRACT

A new cultivar of Styrax plant named JLWEEPING that is characterized by the combination of a weeping habit, pink flowers and leaves which are slightly larger, glossier and less pubescent than other known varieties.

### GENUS

Styrax

### SPECIES

Japonicus

### VARIETAL DENOMINATION

JLWeeping

### BACKGROUND OF THE INVENTION

The present invention relates to a new and distinct cultivar of styrax, or weeping Japanese snowbell that is grown for use as a small tree. It is known botanically as styrax japonica and will be referred to hereinafter by the cultivar name JLWEEPING. The plant was discovered as a seedling of unknown parentage growing in a cultivated area in Salem, Oreg.

The new variety is distinguished from known Japanese snowbell varieties because of the following combination of characteristics: the growth habit is weeping in form and differs significantly from the species which is rounded to broad-rounded with a horizontal branching appearance. While its shape is similar to another weeping cultivar, “fragrant fountains” it forms pink flowers which are slightly fragrant. Additionally, it forms leaves which are slightly larger in length and width, are darker green and glossy, and whose undersides have pubescence only along the veins.

Plants of the new variety have been asexually reproduced in Salem, Oreg., USA, through the use of softwood cuttings or graftings on styrax japonica understock. The characteristics of the new variety have been found to be strictly transmittable by such asexual repropagation, and the new cultivar reliably reproduces in a true to type manner from one generation to another.

### BRIEF SUMMARY OF THE INVENTION

The following traits have been repeatedly observed and represent the characteristics of the subject variety. These traits in combination distinguish the variety from other varieties known to the inventor.

The subject plant exhibits a combination of a weeping habit, pink flowers, and leaves which are slightly larger, glossier, and less pubescent than other known varieties.

Plants of the subject variety have not been observed under all possible environmental conditions. Accordingly, it is possible that phenotypic expressions may vary somewhat with changes in light intensity and duration, cultural practice, and other environmental conditions.

### BRIEF DESCRIPTION OF THE SEVERAL VIEWS OF THE DRAWINGS

FIG. 1 is a close-up view of a 4 year old specimen of the new variety, top grafted on a 36″ standard

FIG. 2 is the same 4 year old, top grafted specimen, but from a distance to show the pendulous habit of the new variety

FIG. 3 is the leaves, flower and flower bud of the new variety

FIG. 4 is a close view of the flowers of the new variety.

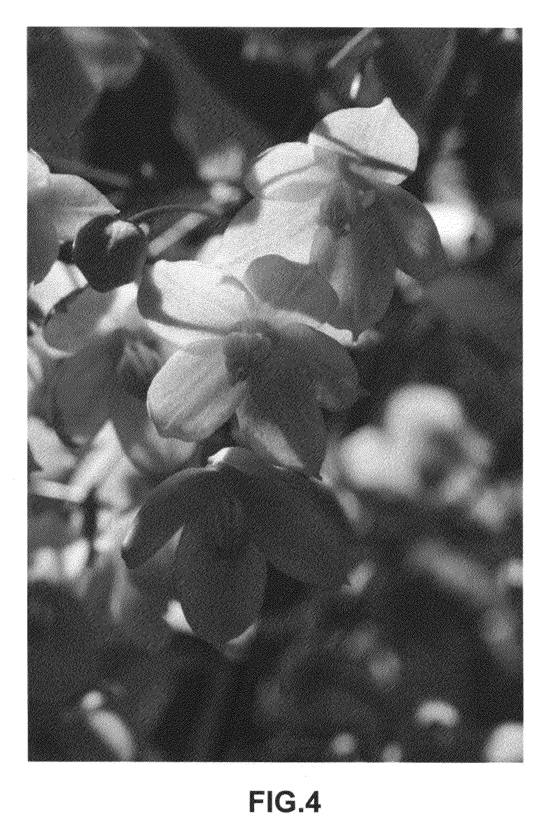

### DETAILED BOTANICAL DESCRIPTION OF PREFERRED EMBODIMENT

The chart used in the identification of color is the R.H.S. Color Chart of The Royal Horticultural Society, London, England. Other reference to color is to be accorded its ordinary dictionary significance. The description is based upon the observation of plants of the new variety having an age of approximately 4 years while being grown outdoors at Salem, Oreg., U.S.A.

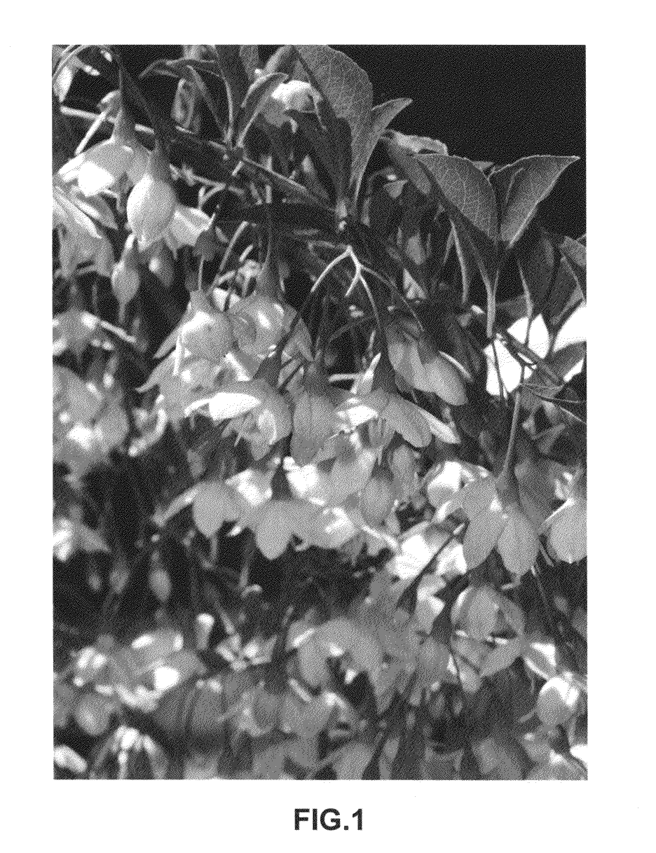

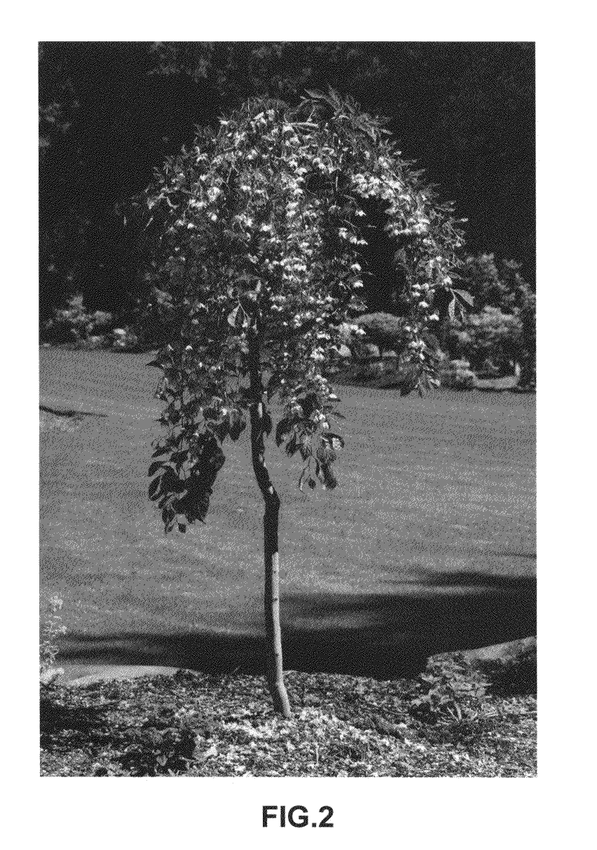

The new variety exhibits extremely pendulous growth habit with a medium growth rate as shown in FIGS. 1 and 2 in this regard.

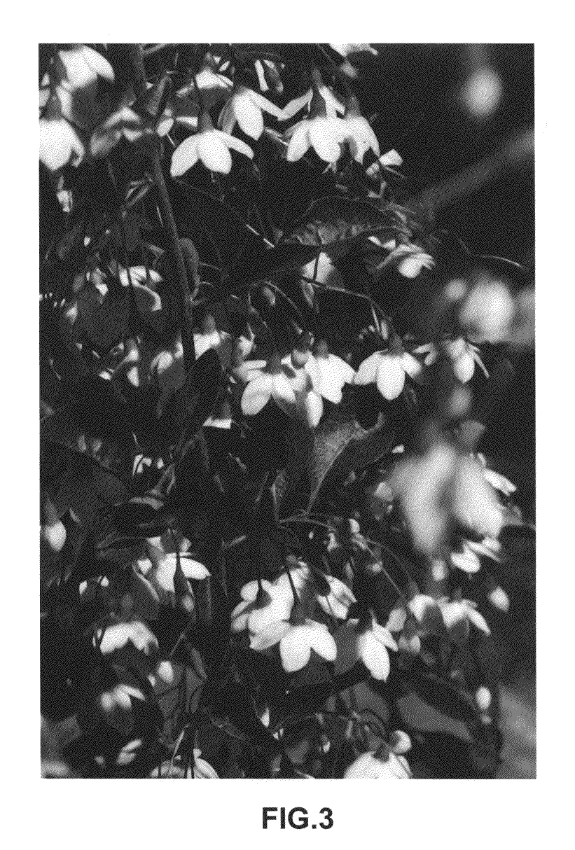

The vegetative parts of the new variety are typical of the genus except as specifically indicated hereafter. The alternate, simple leaves are broadly elliptical with serrated margins, an acuminate apices and cuneate base. See FIG. 3 in this regard. The petiole is approximately ⅜″ in length compared to the typical ⅓″ of the species. The species leaves are normally 1 to 3½″ long and ½ to 1½″ wide. The new variety has leaves approximately 3½ to 4″ in length and around 2 to 2½″ in width. Compared to another weeping variety, ‘Fragrant Fountains’, its leaves are much larger, darker in color and glossier. ‘Fragrant Fountains’ has a light pubescence along the entire underside of its leaves unlike the new ‘JLWeeping’ variety which only has pubescence along the veins.

The emerging leaves of the new variety in the spring on the upper surface are like Green Group 144A. As leaves mature, they deepen to Green Group 139A with an underside of Green Group 137C.

Immature twigs, mature branches and bark are all typical in color to that of the species and do not differ significantly or increase the ornamental value of the tree over other varieties.

The blossom appearance of the new variety is similar to that of the genus. The new variety forms in profusion attractive pink blossoms which, while in bud generally correspond to the color of Red-Purple Group 65A, opening to Red-Purple Group 65 B, C & D. The bloom stem ranges in color from Red-Purple Group 60A, B & C.

The overall configuration of the flowers is generally typical of the genus. The flowers are perfect, pink, bell-shaped and numerous. Blossoming commonly occurs during mid-May to Mid-June at Salem, Oreg., U.S.A., where it commonly extends over approximately 3 to 4 weeks depending upon the weather conditions that are encountered. The flowers are commonly borne on a 1 to 1½″ long pendulous stalk, occurring on short lateral shoots, each shoot has multiple flowers which are lightly fragrant. As shown in FIGS. 3 & 4, the blossoms are corolla 4-6 lobed being united near the base, with the margins having a slight ruffle at times. The reproductive organs are typical of a Styrax japonica and other species and cultivars angiosperm flowering plant. The anthers are Yellow Group 15A in coloration.

During observations to date, fruit has been formed and is not considered to be ornamental in nature. It forms a dry ovoid, about ½″ long, apparent in August and falling around November which is typical of the species.

When grown at Salem, Oreg., U.S.A., the new variety has exhibited disease resistance under observations to date and has not been affected to any degree by common Styrax diseases or by insects which commonly attack Styrax. The new variety has proven to be hardy when tested in U.S.D.A. Hardiness Zone 5.

The terms and expressions which have been employed in the foregoing specification are used therein as terms of description and not of limitation, and there is no intention, in the use of such terms and expressions, of excluding equivalents of the features shown and described or portions thereof, it being recognized that the scope of the invention is defined and limited only by the claims which follow.

What is claimed is:

1. A new and distinct cultivar of styrax plant named JLWEEPING, as described and illustrated herein.
